(19) United States
(12) Patent Application Publication
(10) Pub. No.: US 2016/0030710 A1
(43) Pub. Date: Feb. 4, 2016

(54) Reduced cavitation in catheter removal

(71) Applicant: John T.R. Wilson, Seattle, WA (US)

(72) Inventor: John T.R. Wilson, Seattle, WA (US)

(21) Appl. No.: 14/756,602

(22) Filed: Sep. 23, 2015

Related U.S. Application Data
(63) Continuation of application No. 13/998,397, filed on Jan. 13, 2014.

(51) Int. Cl. A61M 25/00

(52) U.S. Cl. CPC A61M 25/0043; A61M 25/007; A61M 2025/0062; A61M 2025/0059

(57) ABSTRACT

This invention concerns urinary catheterizing and identifies a cavitation occurring at the moment of catheter removal after catheterizing drainage is completed as the source of a transient damaging problem, and shows that venting to atmosphere of the catheter tip prior to the catheter removal prevents or reduces this transient damaging problem. The venting to atmosphere prevents a transient and partial vacuum formation for a transient cavitation. The invention disclosure shows the vent and method for preventing or reducing this transient cavitation damage.

The prior art for self-catheterizing specifies six catheterizings per day. Thus, the prior self-catheterizing art specifies the catheter removals that precede the transient damaging problem. Although each catheter removal causes a small amount of damage, the repeated damage is cumulative and enlarges as subsequent catheter insertions disrupt the healing of previous damage. After many catheterizings, the transient cavitation damage at catheter removal leads to a serious bleeding problem.

### CROSS REFERENCE

This application is a continuation of prior Non-Provisional application Ser. No. 13/998,397, filed Jan. 13, 2014 entitled “Cavitation Reduction in Catheter Removal”.

### BACKGROUND OF THE INVENTION

This invention relates to the human urinary track and to draining urine from the bladder by catheterizing. Such catheterizing sometimes causes bleeding. Certain urinary track bleeding related to catheterizing is the problem solved by this invention.

Cavitation is a hydraulic term, often used in pump design and action, meaning the formation of a partial vacuum in a liquid and the subsequent collapse of the vacuum causing cavitation damage to solid surfaces. This invention identifies cavitation as a transient damaging problem in urinary catheterizing at the moment of catheter removal after catheterizing drainage is completed. This invention shows venting to atmosphere of the catheter tip prior to catheter removal prevents a transient partial vacuum formation of cavitation and thus prevents transient cavitation and the transient damage of this cavitation on solid surfaces enclosing or interfacing with this catheterizing. The prior catheterizing art gets cavitation damage at catheter removal; then, the next catheter insertion disrupts any healing from the previous catheter-removal cavitation damage. Thus, the prior catheterizing art leads, after many catheterizings, to serious urinary bleeding. The self-catheterizing art specifies six to seven catheterizings per day.

In human catheterizing, bleeding is undesirable from any source in the urinary track. Sharp edges on the catheter can cause bleeding. Catheterizing without allowing sufficient time for healing of previous bleeding sources is undesirable.

Biopsies of bladder cells are done to identify bleeding sources. Biopsies have identified impact-damaged bladder cells as suspected bleeding sources.

### BRIEF SUMMARY OF THE INVENTION

The invention recognizes that damaging urinary cavitation may occur after human bladder catheterizing upon removal of a configured catheter, considered 17 inches long, and describes a method and devices to reduce or avoid that cavitation. This invention describes an apparatus and a method to reduce or avoid cavitation occurring at the tip of a 17 inch catheter containing residual urine and progressing into a configured urinary parts interface by venting air or other fluid to the tip of the configured catheter prior to or coincident with or during removal of the configured catheter following catheterizing. By admitting the atmospheric or outside air or other fluid to the tip of the configured catheter, no partial vacuum will be formed when the catheter is removed from the configuring urethra. The vent is 22 inches long.

### BRIEF DESCRIPTION OF THE DRAWINGS

This invention is described using eight Figures and include some enlargements for ease of presentation. FIG. 1 through FIG. 6 depict cross-sections through a configured urinary parts interface in various stages of catheterizing. The configured urinary parts interface is considered to include: urethra 2, sphincter 5, valve 6, bladder 4, left-kidney ureter 7 and right-kidney ureter 8. FIG. 7 depicts a catheter. FIG. 8 depicts a vent line. The FIG. 1 and FIG. 2 depict the initial stages of catheterizing; these FIGS. 1 and 2 are considered Prior Art. The FIG. 3 and FIG. 4 depict stages of catheterizing following the FIG. 2 stage without using this invention; these FIGS. 3 and 4 are considered Prior Art. The FIG. 5 and FIG. 6 depict stages of catheterizing following the FIG. 2 stage using this invention.

FIG. 1 depicts bladder 4 being drained of urine 10 through catheter 1 with the valve 6 open. The catheter 1 is shown after being inserted into the urethra 2, using lubricant 3 and pushing valve 6 to open to the bladder 4. This FIG. 1 shows prior art.

FIG. 2 depicts sphincter 5 operated to close valve 6 to interrupt the flow path from the bladder 4 to the catheter 1 thus stopping the flow of urine 10. This FIG. 2 shows prior art. FIG. 2 also depicts some of configured urinary parts interface 9 between valve 6 and catheter 1.

FIG. 3 depicts the stage following the FIG. 2 stage without using this invention. FIG. 3 depicts urine 10 and the formation of a partial vacuum 11 in the configured urinary parts interface 9 and elsewhere at the start of catheter 1 removal. The partial vacuum 11 extends from the configured urinary parts interface 9, through the valve 6 and into the bladder 4.

FIG. 4 depicts the stage that follows the FIG. 3 stage without using this invention. FIG. 4 depicts the collapse of the partial vacuum 11 into pieces trapping urine 10 drops within the vacuum pieces 11 and impacting these pieces, onto solid surfaces of bladder 4. valve 6 and ureters 7 and 8. FIG. 4 is prior art.

FIG. 5 depicts the stage following the FIG. 2 stage using this invention. FIG. 5 also depicts this invention with the introduction of vent 12 into the flow path of catheter 1 to the tip of catheter 1. The vent 12 is a flexible plastic line 22 inches long of 0.065 inches diameter. FIG. 5 also depicts the sphincter 5 operated to close valve 6 at catheterizing completion to interrupt the flow path from the bladder 4 to the catheter 1 thus stopping the flow of urine. FIG. 5 also depicts the introduction of atmospheric air 13 through and around the vent 12 at catheterizing completion.

FIG. 6 depicts the stage following the FIG. 5 stage using this invention. FIG. 6 also depicts the catheter 1 partially removed from the configured urinary parts interface 9 with no partial vacuum formation, nor vacuum collapsing, nor urine 10 in vacuum pieces, impacting solid surfaces of bladder 4, valve 6, urethra 2, or ureters 7 and 8.

From the stage depicted in FIG. 6 to complete one catheterizing, the configured catheter 1 and vent 12 would be simultaneously fully withdrawn from the configuring urethra 2 having suppressed any transient partial vacuum formation, transient cavitation, or cavitation damage.

FIG. 7 depicts a catheter 1 in approximately full size. The tip of the catheter 1 is to the left and has the rounded point. The tip is inserted in the urethra 2 in catheterizing, and in FIG. 7 the tip has the elongated opening outline, which is the entry for urine from the bladder 4. During catheterizing, the outlet of urine from the catheter during catheterizing is from the squared end. The length of the catheter 1 is considered to be 16 to 17 inches.

FIG. 8 depicts a vent 12 in approximately full size. The length of the vent 12 is considered to be 22 inches.

### DETAILED DESCRIPTION OF THE INVENTION

The invention will be described by describing the drawings in more detail in a catheterizing sequence.

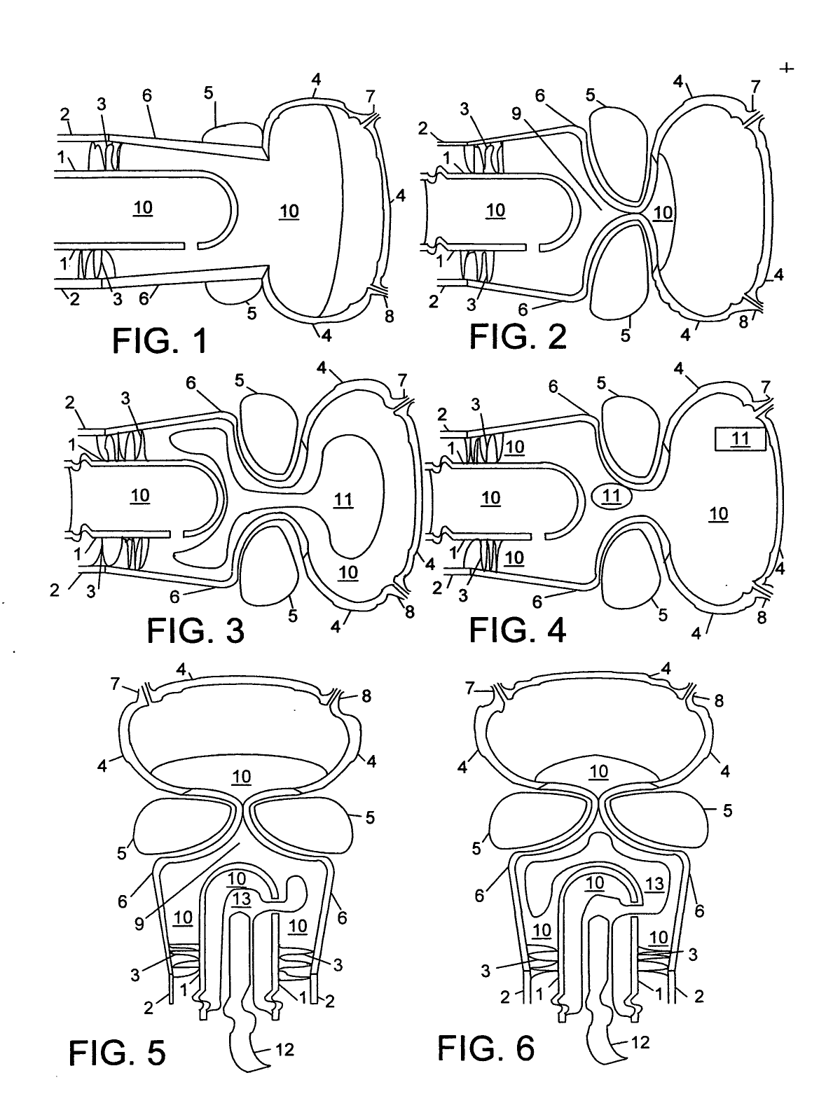

In FIG. 1 is depicted catheterizing in the process of draining urine 10 from the bladder 4 into the catheter 1 via the hole in catheter 1 in the inlet of catheter 1. Catheterizing starts when the catheter 1 and the mouth of urethra 2 have been coated with lubricant 3 and the catheter 1 has been inserted up the urethra 2 to push open the valve 6 and start flow of urine 10. FIG. 1 is prior art.

In FIG. 2 is depicted catheterizing in which the draining of urine 10 from bladder 4 has been determined to be completed and the sphincter 5 has constricted and closed valve 6. A small amount of urine 10 remains in bladder 4 and some urine 10 remains in the tip of catheter 1 and for some distance to the left in catheter 1. FIG. 2 is prior art.

The FIG. 3 depicts the stage of catheter removal without the use of subject invention following the FIG. 2 stage. The FIG. 3 shows catheter 1 slightly withdrawn to the left causing the transient formation of a partial vacuum 11 in the configured urinary parts interface 9, previously shown in FIG. 2, and the partial vacuum 11 permeates the urine 10. FIG. 3 is prior art.

The FIG. 4 depicts the stage following the FIG. 3 stage which includes the transient expansion of the transient partial vacuum 11 through valve 6 and into bladder 4. The transient partial vacuum 11 collapses into pieces, depicted as rectangular and spherical, next to valve 6, bladder 4 and ureters 7 and 8. The urine 10 permeates the transient partial vacuum 11 pieces and as those transient vacuum pieces 11 collapse with urine 10 drops inside, the urine 10 drops makes damaging impacts on enclosing or interfacing solid surfaces. This is called cavitation. This is what this invention prevents.

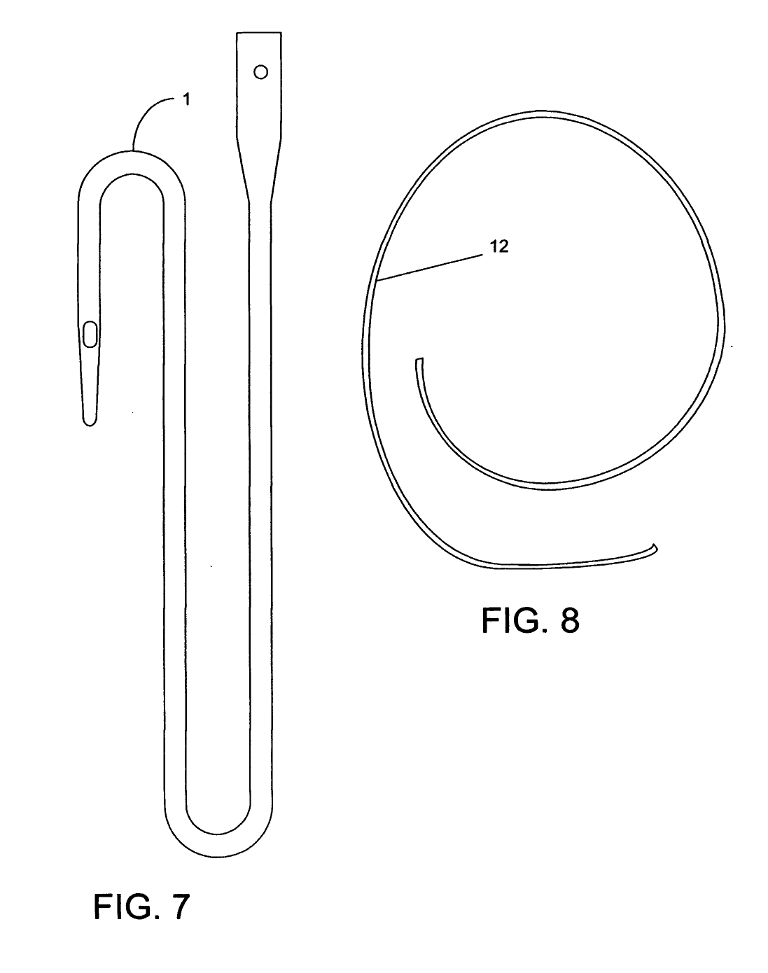

In FIG. 5 is depicted the stage following from the FIG. 2 stage using this invention, in which the vent 12 of this invention is introduced into the catheter 1. The vent 12 is a flexible plastic line which may be a tube. This vent 12 is introduced into the discharge end of catheter 1, shown as the squared end in FIG. 7, and pushed to the tip of catheter 1. The stiffness of vent 12 is enough to allow the vent 12 to be pushed in the catheter 1. By this action the atmospheric air 13 is introduced to the tip of catheter 1 and into the configured urinary parts interface 9. The presence of atmospheric air at the tip of catheter 1 prevents the formation of a partial vacuum at this place and in the surrounding place. The vent 12 used is a flexible plastic line longer than the catheter 1 for ease of manual manipulation and of a diameter to fit easily within the catheter 1 flow path. The ends of the vent 12 are filed to prevent gouging the inside diameter of the catheter 1.

The FIG. 6 depicts the stage following from the FIG. 5 stage using this invention. The FIG. 6 shows the catheter 1 and vent 12 slightly withdrawn from the configured urinary parts interface 9 causing a vacancy in the urinc 10 in the configured urinary parts interface 9 which vacancy is filled by atmospheric air 13; at this configured catheter and vent line withdrawal from configured urethra 2 there is no partial vacuum 11 formed and there is no cavitation nor the cavitation caused damage.

From the stage depicted in FIG. 6, to complete one catheterizing, the configured catheter 1 and vent 12, together and simultaneously, are fully withdrawn from the configuring urethra 2.

A working model of this invention has been made and tested. The catheter 1 has been a Bard 16 Fr. Caude tip, 16 inches long. The vent 12 has been an Arnold Trimmer line, 0.065 inches diameter and 22 inches long; the ends have been filed to prevent gouging the inside diameter of the catheter.

While certain preferred embodiments of the present invention have been disclosed in detail, it is understood that various modifications in its structure may be adopted without departing from the spirit of the invention or the scope of the following claims.

I claim as my invention:

1. A urinary catheter wherein the improvement comprises reduction of urinary transient-cavitation vacuum-collapse damage at a completed-catheterizing catheter removal from a configured catheter urethra by inserting an independent vent line to the configured catheter tip followed by the simultaneous removal of both the configured catheter and the independent vent line from the configured urethra.

2. A catheter as in claim 1 wherein the configured catheter and independent vent line simultaneous removal is done after insertion of the independent vent line.

3. An apparatus for removing human urine via a configured catheter inserted through a lubricated urethra, to a configured urinary parts region comprising a bladder and sphincter valves and ureters, and an independent vent line, for reducing cavitation generation, said independent vent line inserted through the configured catheter discharge lumen to the configured catheter tip at catheterizing drainage completion to vent the configured urinary parts region.

4. An apparatus as in claim 3 wherein the independent vent line is separate from the configured catheter and of a length sufficient to extend from the configured catheter lumen for urine discharge to the configured catheter tip and to be controllable over the configured catheter length by a surplus length of independent vent line length over the configured catheter length.

5. An apparatus as in claim 4 wherein the independent vent line is of a diameter that fits movably within the configured catheter urine-flow channel diameter.

6. An apparatus as in claim 5 wherein the independent vent line is inserted to the configured catheter urine flow discharge lumen prior to the simultaneous removal of the configured catheter and the independent vent line from the configured urinary parts region.

7. A method of catheterizing comprising admitting air or other fluid to a configured urinary parts region prior to or coincident with configured catheter removal to prevent or partially reduce transient vacuum formation in the configured urinary parts region to prevent or reduce cavitation damage.

8. A method of catheterizing as in claim 7 wherein the prevention or reduction of vacuum formation is to prevent or reduce cavitation damage when the vacuum collapses.
